(19) United States
(12) Patent Application Publication
(10) Pub. No.: US 2022/0361788 A1
(43) Pub. Date: Nov. 17, 2022

(54) SYSTEM AND METHOD FOR MEASURING ACUTE AND CHRONIC STRESS

(71) Applicant: Mahir Shah, Fort Mill, SC (US)

(72) Inventor: Mahir Shah, Fort Mill, SC (US)

(21) Appl. No.: 17/302,743

(22) Filed: May 11, 2021

(51) Int. Cl. A61B 5/16; A61B 5/024; A61B 5/08; A61B 5/00; A61B 5/0205; G16H 50/30

(52) U.S. Cl. CPC A61B 5/165; A61B 5/024; A61B 5/0816; A61B 5/4803; A61B 5/0205; A61B 5/0002; G16H 50/30; A61B 5/163; A61B 5/14546

(57) ABSTRACT

Examples of the present subject matter provide techniques for measuring stress levels using a variety of physiological indicators, such as pupil diameter, voice, cortisol levels, skin resistance, etc. Different sensors may be provided to measure the physiological indicators. Those measurements may be collected at a central server, where those measurements may be analyzed to determine the stress level of the user.

### TECHNICAL FIELD

The present disclosure generally relates to measurement of stress and other behavioral conditions, particularly acute and chronic stress for clinicians.

### BACKGROUND

Stress can negatively impact a person's wellbeing. This impact is especially felt by health care providers, such as doctors, clinicians, nurses, etc. Medicine can be an extremely stressful profession. Indeed, the suicide rate for health care providers is significantly greater than the general population.

The source of the stress for health care providers can range from job responsibilities to individual patients. Before receiving treatment for stress related issues, it may be helpful to determine the level of stress along with the source of the stress.

### BRIEF DESCRIPTION OF THE DRAWINGS

Various ones of the appended drawings merely illustrate example implementations of the present disclosure and should not be considered as limiting its scope.

FIG. 1 illustrates a block diagram of a stress monitoring system in accordance with an embodiment of the present subject matter.

FIG. 2 illustrates a watch in accordance with an embodiment of the present subject matter.

FIG. 3A illustrates a pupil sensor in accordance with an embodiment of the present subject matter.

FIG. 3B illustrates a pupil sensor in accordance with an embodiment of present subject matter.

FIG. 4 illustrates a user device in accordance with an embodiment present subject matter.

FIG. 5 illustrates an EHR server in accordance with an embodiment of the present subject matter.

FIG. 6 illustrates a graphical representation of a stress reading in accordance with an embodiment of the present subject matter.

FIG. 7 illustrates a flow diagram of a method to generate a stress reading in accordance with an embodiment of the present subject matter.

FIG. 8 illustrates a flow diagram of a method to calculate a stress reading during an abnormal condition in accordance with an embodiment of the present subject matter.

FIG. 9 illustrates a block diagram of an example comprising a machine upon which any one or more of the techniques (e.g., methodologies) discussed herein may be performed.

### DETAILED DESCRIPTION

The inventor has recognized a need in the art for systems and methods to measure stress levels in different environments. Examples of the present subject matter provide techniques for measuring stress levels using a variety of physiological indicators, such as pupil diameter, voice, cortisol levels, skin resistance, etc. Different sensors may be provided to measure the physiological indicators. Those measurements may be collected at a central server, where those measurements may be analyzed to determine the stress level of the user. In an example, the physiological measurements may be taken of a health care provider while that health care provider is tending to a patient. Hence, the data collected may reflect stress levels that can be analyzed and categorized by different events in the health care provider's schedule.

Moreover, central server may be implemented by an Electronic Health Record (EHR) system. The EHR system may provide information regarding stress levels and may calculate different stress measurements. The EHR system may calculate custom individualized stress measurements based on historical data for the user. Moreover, the EHR system may provide notifications and resources for the user. Thus, the EHR system may able to connect the user with resources to assist with their stress.

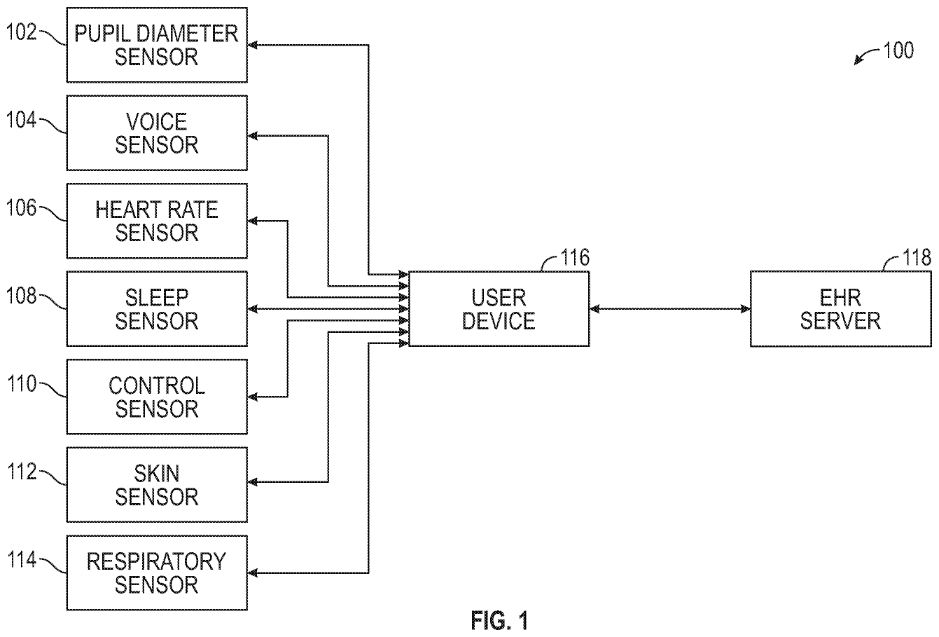

FIG. 1 illustrates a block diagram of a stress monitoring system 100 in accordance with an embodiment of the present subject matter. The stress monitoring system 100 may include a pupil diameter sensor 102, a voice sensor 104, a heart rate sensor 106, a sleep sensor 108, a cortisol sensor 110, a skin sensor 112, a respiratory sensor 114, a user device 116, and an EHR server 118.

The pupil diameter sensor 102 may measure a pupil diameter of a user. The pupil diameter sensor 102 may be provided as a pupilometer. The pupil diameter sensor 102 may include infrared sensors to electronically measure the pupil diameter of user. The size of the pupil tends to increase with stress.

In another embodiment, the pupil diameter sensor 102 may be provided as an optical sensor. The optical sensor may capture an image of a user's eye and may use an algorithm to calculate the diameter of the user's pupil.

The voice sensor 104 may measure a frequency and/or decibel level of the user's voice. Voice frequency tends to get higher with stress. The voice sensor 104 may be provided as a microphone. The voice sensor 104, for example, may be incorporated into a wearable device, such as a watch. The voice captured by the microphone may be measured to determine the frequency and/or decibel level of the user's voice. Speech is a psychophysiological process. By measuring voice properties, such as microtremor, fundamental frequency, syllable increase or reduction, vowel duration, word intensity, respiratory variability, etc., emotional state of the user may be determined. Speech is regulated by higher brain regions and thus allows detection and differentiation between emotional loads in addition to physiological changes in the human body. Voice analysis may allow differentiation between cognitive and emotional load/stress. For instance, anger and irritability correlate with increase vowel duration and increased word intensity.

Voice analysis may also help in detecting elevated high levels of drugs such as downers, alcohol, date rape drugs, benzodiazepines, etc. Thus, in an embodiment, the device incorporating the voice sensor 104, such as a wearable device, may detect change in speech patterns and may generate an alert to warn the user of possible elevated levels of drugs.

The heart rate sensor 106 may measure the heart rate and/or heart rate variability of the user. Heart rate tends to increase with stress. The heart rate sensor 106 may be provided as a chest monitor worn around the chest of the user. The chest monitor may generate electrical signals responsive to detected heart beats and may transmit corresponding electrical signal to a receiver. In another embodiment, the heart rate sensor 106 may be provided as an optical sensor for detecting variations in vessels and respiratory rate, which is indicative of the user's heart rate. In yet another embodiment, the heart rate sensor 106 may be provided using photoplethysmography (PPG) sensor(s), which employ light sensors to detect the rate of blood flow as controlled by the pumping rate of the heart.

The heart rate sensor 106 may also be incorporated on a wearable device, such as a watch. The heart rate sensor 106 may include electrocardiogram (EKG) sensors. The heart rate sensor 106 may detect R-R interval or inter beat interval (time between beats measured in milliseconds). These interval detections may allow measurement of the temporal difference between successive heart beats.

Heart rate variability can be an indicator of stress. Increasing levels of stress can result in a decrease in the high frequency and an increase in the low frequency of the R-R interval.

The heart rate sensor 106 may also detect absolute heart rate. Heart rate can also be a reliable indicator of stress but may not be as sensitive as heart rate variability. As discussed below, heart rate measurements may be excluded in the overall assessment of stress level while the user is vigorously exercising and sometime thereafter (e.g., 45 minutes). User's exercising may be detected by an accelerometer worn by the user, for example in a watch.

A sleep sensor 108 may monitor sleep levels of the user. The sleep sensor 108 may utilize actigraphy to measure the user's rest and activity cycles. The sleep sensor 108 may be provided as an accelerometer for detecting the motion, or lack of motion, of the user. The sleep sensor 108 may utilize a microphone to detect motion, detecting quality of sleep (e.g., snoring, restless sleep).

Deep sleep is a time when most physiological parameters may be at their lowest, so it may be a good indicator of a baseline for the parameters and stress. As discussed below, a personalized baseline for stress level (including measured physiological parameters) may be determined for a user.

Moreover, stress and anxiety can lead to insomnia and other sleep problems. Conversely, lack of proper rest can contribute to stress. Because of this reciprocal relationship, addressing one of these issues (stress or rest) can lead to improvement of the other. Thus, sleep parameters may be detected, such as sleep onset, maintenance, and consolidation. From these detected sleep parameter(s), information about sleep stages may be determined as well. And severity of sleep deprivation may be determined and incorporated into the overall stress assessment, as described below.

A cortisol sensor 110 may measure cortisol levels of the user. The cortisol level may be an indicator of a user's stress level and is considered a stress hormone.

The skin sensor 112 may measure the user's skin conductance or electrical resistance. Electrical resistance of the skin tends to decrease with stress. For example, the skin sensor 112 may measure the galvanic skin response of the user. That is, it may measure the skin conductance via change in electrical resistance. Galvanic skin response (GSR) or electrodermal activity is also known as Psychogalvanic reflex. GSR can be one of the most sensitive and accurate markers of emotional arousal. GSR, may not be representative of the type of emotion but the intensity of it. Increase in stress may be reflected by decreased electrical resistance due to increase in sweat and electrolytes, which may be measured, for example, on the wrist.

The skin sensor 112 may also include a skin temperature sensor to detect the surface temperature of the skin. The skin temperature sensor may be calibrated so as to compensate for the effects of outside variables, such as ambient temperature, sunlight, exercise being performed, etc. The skin temperature sensor may detect whether the user is febrile (showing symptoms of having a fever). As described below, detection of a fever may lead to excluding the corresponding period from stress analysis because a fever can affect the physiological factors indicating stress and in fact can cause increased stress.

The respiratory sensor 114 may measure the respiratory rate of the user. For example, the respiratory sensor 114 may use a PPG sensor to measure the respiratory rate. The respiratory sensor 114 may include an oxygen level sensor. Respiratory variation can be an indicator of emotional, cognitive and behavioral demands/stress. Different properties of respiratory rate may be measured, such as respiratory variability, sighing, one breath lag, minute ventilation, tidal volume, inspiratory and expiratory time, etc. These respirator properties may provide indicators of stress level, as further described below. For example, high stress readings related to drop in oxygen level may trigger a warning to the user.

Alternatively, or additionally, other sensors may also be provided. For example, a pulse sensor may measure the pulse of the user. A perspiration sensor may measure the perspiration (sweat) level of the user.

A blood pressure sensor may measure the blood pressure of the user. The blood pressure sensor may be provided as an optical sensor. The human body produces a surge of hormones when in a stressful situation. These hormones temporarily increase blood pressure by causing the heart to beat faster and the blood vessels to narrow. These spikes may be measured by an optical blood pressure monitor, for example on a watch. Substantial cardiovascular responses to acute psychological stress as opposed to acute physical exertion may be considered pathophysiological. Thus, as further explained below, blood pressure and/or heart rate elevation due to psychological stress as opposed to acute physical exertion (e.g., exercise) may be distinguished.

Blood pressure and heart rate baselines can vary amongst individuals. For example, a blood pressure of 120/80 and pulse of 72 beats per minute are considered to be in the normal range, but in some cases, these factors may be lower or higher for some individuals. For example, blood pressure medication can have an effect on readings of blood pressure and resting heart rate. Non-compliance with medications may affect readings as well (but even in those cases, spikes related to stress may still be detected). Some people may suffer from untreated hypertension, affecting these readings, too. Also, higher fitness levels can correlate with a lower resting heart rate. Thus, as described below, a personalized baseline of these and other parameters may be determined for an individual in non-stressful situations.

Other examples of sensors may include an oxygen sensor for measuring oxygen levels of the user (e.g., amount of oxygen consumed) and an EKG monitor for measuring electrical signals from the heart of a user.

The sensors (e.g., 102-114) may be provided in a variety of configurations. As discussed in further detail below, a set of sensors (e.g., the voice sensor 104, the heart rate sensor 106, the sleep sensor 108, the skin sensor 112, the respiratory sensor 114) may be integrated into a smart watch. Other sensors (e.g., pupil diameter sensor 102, voice sensor 104) may be integrated into a computer, tablet, laptop, or the like.

The sensors 102-114 may measure physiological changes and report those changes to the user device 116. The user device 116 may be provided as a computing device such as a computer, laptop, tablet, etc. The user device 116 may also be provided as mobile devices including, but not limited to, mobile smartphones, such as Android® phones and iPhones®, tablets, or hand-held wireless devices such as PDAs, multiprocessor systems, microprocessor-based or programmable consumer electronics, minicomputers, other handheld devices that may now be available or may in the future become available. The user device 116 may utilize a downloadable app supplied by the EHR system for use in conjunction with the EHR server 118.

In an embodiment, the sensors 102-114 may communicate with the user device 116 via short range communication using Bluetooth, Zigbee, IrDa or other suitable protocol. In another embodiment, some sensors (e.g., pupil diameter sensor 102, voice sensor 104) may be integrated with the user device 116.

The user device 116 may be communicatively connected to the EHR, server 118 via a communication link. The communication link may be provided by one or more networks, such as the Internet. The network may include a wired or wireless local area network (LAN) and a wide area network (WAN), wireless personal area network (PAN) and other types of networks. Computers may be connected over the Internet, an Intranet, Extranet, Ethernet, or any other system that provides communications. Example communication networks may include a local area network (LAN), a wide area network (WAN), a packet data network (e.g., the Internet), mobile telephone networks (e.g., cellular networks), Plain Old. Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others.

The user device 116 may collect information from the sensors 102-112 (and other sensors as described herein) and may send those to the EHR server 118. In an embodiment, the user device 116 may condense or aggregate the information from the sensors 102-114 before sending the sensor information to the EHR server 118. The user device 116 may register the sensor information to the user using an app.

The EHR server 118 may receive the sensor information and may store that information. The EHR server 118 may store the sensor information based on the identity of the user. The EHR server 118 may also perform analysis of the stored information, as described in further detail below. For example, the EHR server 118 may compare the physiological change information to reference levels and generate a display showing the comparison, where the comparison may indicate increased levels corresponding to higher stress levels. The EHR server 118 may also calculate a stress reading based on the physiological change information.

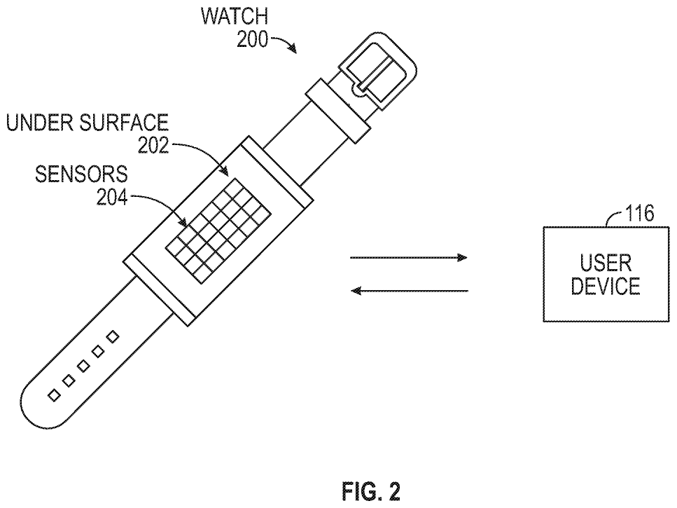

As mentioned above, one or more sensors may be provided on a watch that can be worn by the user. FIG. 2 illustrates a watch 200 in accordance with an embodiment of the subject matter. The watch 200 may include an undersurface 202 and one or more sensors 204. The sensors 204 may be placed on the undersurface 202 so that they are in contact with the skin of the user. The sensors 204 may include a voice sensor (e.g., 104), a heart rate sensor (e.g., 106), a sleep sensor (e.g., 108), a cortisol sensor (e.g., 110), a skin sensor (e.g., 112), and a respiratory sensor (e.g., 114), as described herein. Some sensors may be placed on the top surface of the watch, e.g., voice sensor, sleep sensor.

The watch 200 may collect the information from the sensors 200 and may transmit the information to the user device 116, as described herein. In an embodiment, the watch 200 may transmit the information from the sensors 200 directly to the EHR server 118.

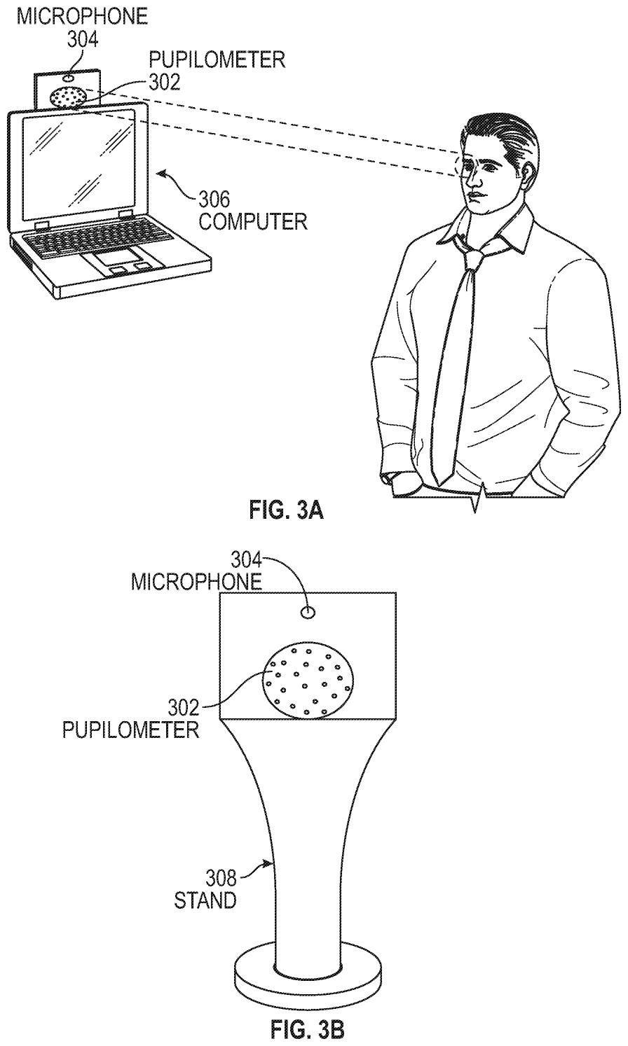

FIG. 3A illustrates a pupil sensor 300 in accordance with an embodiment of the present subject matter. The pupil sensor 300 may include a pupilometer 302 and a microphone 304. The pupil sensor 300 may be attached to a computer 306, which may be a laptop, tablet, or the like. Therefore, the pupilometer 302 may be substantially at eye level of the user as they type notes during a patient visit, for example in an EHR system. The pupilometer 302 may include infrared sensors to electronically measure the pupil diameter of user.

FIG. 3B illustrates another embodiment of pupil sensor 300 in accordance with an embodiment of the present subject matter. The public sensor 300 may also be placed on a stand 308. The height of the stand 306 may be set so that the pupilometer 302 may be substantially at eye level of the user. The stand 308, for example, may be placed behind the patient so that the pupilometer 302 may measure the user's pupil as they examine a patient.

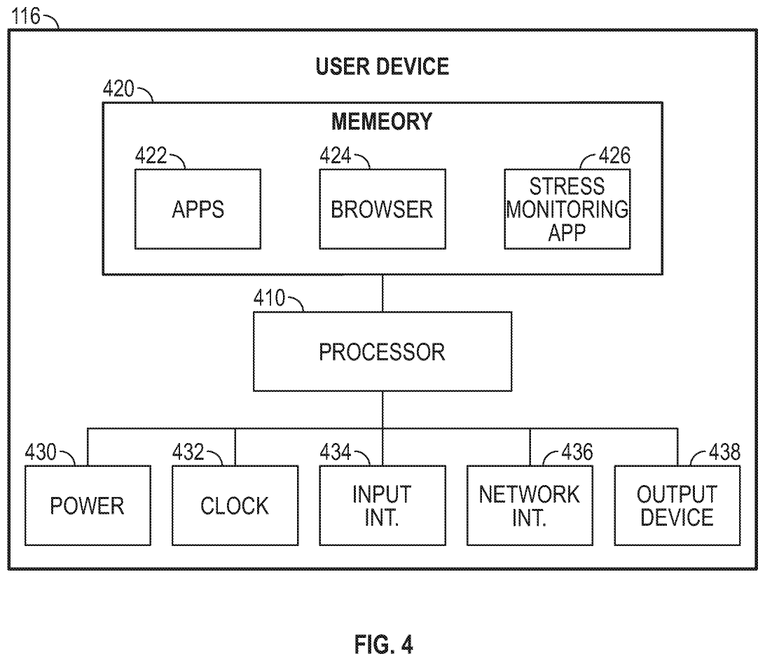

FIG. 4 illustrates a user device 116 in accordance with an embodiment of the present subject matter. The user device 116 may include a processor 410 and a memory 420. The memory 420 may include, for example, applications 422, a browser 424, and a stress monitoring application 426. The processor 410 may also be connected to additional components, either stored in a memory or installed as separate hardware components, such as a power source 430, a clock 432, an input interface 434, a network interface 436, and output devices 438.

In embodiments of the present subject matter, users may be required to subscribe to the EHR in order to use the stress monitoring application 426. The EHR may store medical files and other health care related information.

The user device 116 may interact with EHR system using a browser 424 to access an electronic trailing system website as will be further described below. Alternatively, the user device 116 may interact with the EHR system using a mobile application. In embodiments of the present subject matter, the stress monitoring application may cause the user to access one or more URLs from the EHR website. In an embodiment, the stress monitoring application may provide the ability to automatically take actions based on notifications or other pushed data.

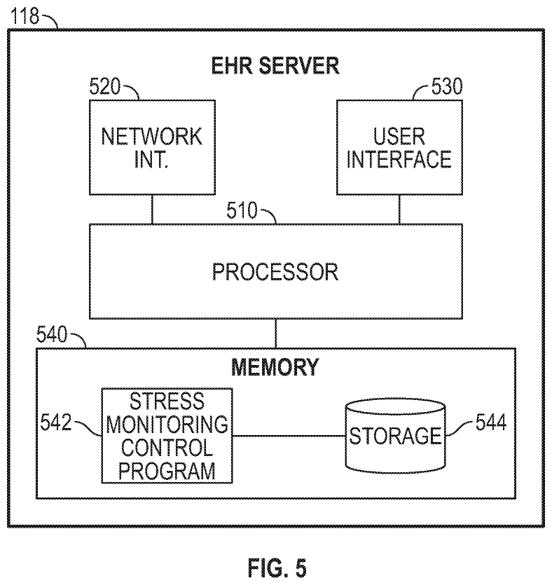

FIG. 5 illustrates an EHR server 118 in accordance with an embodiment of the present subject matter. The EHR server 118 may include a processor 510, a network interface 520, a user interface, 530, and a memory 540. The network interface 520 may be used for communicating with the user device 116 and other systems over a network, and the user interface 530 may allow viewing and input directly by a vendor of the EHR system. The memory 540 may include a stress-monitoring control program 542 connected with a storage element 544. The processor 510 may execute various analysis algorithms from the stress-monitoring control program 542 as will be further described below. The storage element 544 may store data gathered from the various sources described above with respect to FIG. 1, such as from user devices 116. This data may be structured, semi-structured, or unstructured. The data storage areas may include file systems and databases for storing large amounts of data. Data stored in the storage element 544 may be managed and communicated with an Object-Relational Database Management System, as known in the art. The storage element 544 may include multiple data storage areas, which may have different structures and store different types of data. For example, unstructured data may be stored separately from cleansed and structured data.

The EHR server 118 may collect the sensor information from the user, analyze it, and display results for the user to access. The EHR server 118 may display individual parameters on an aggregate scale. The scale for the individual parameters may be compared to average values for healthy subjects in same age group or to personalized baseline values, as described in further detail below. For example, the EHR server 118 may compare the sensor information against reference baselines. For example, normal respiratory rate is approximately 12-16 breaths a minute Thus, the EHR server 118 may extract the respiratory rate of the user from the received sensor information, compare it to the reference value, and display the result for the user to access, for example, from the app. The result may be displayed in graphical representation and/or a numerical representation.

The EHR server 118 may also generate a stress reading based on the received sensor information (e.g., physiological measurements). The stress reading may be a combination of the different physiological measurements with appropriate weights. The stress reading may be displayed in graphical form.

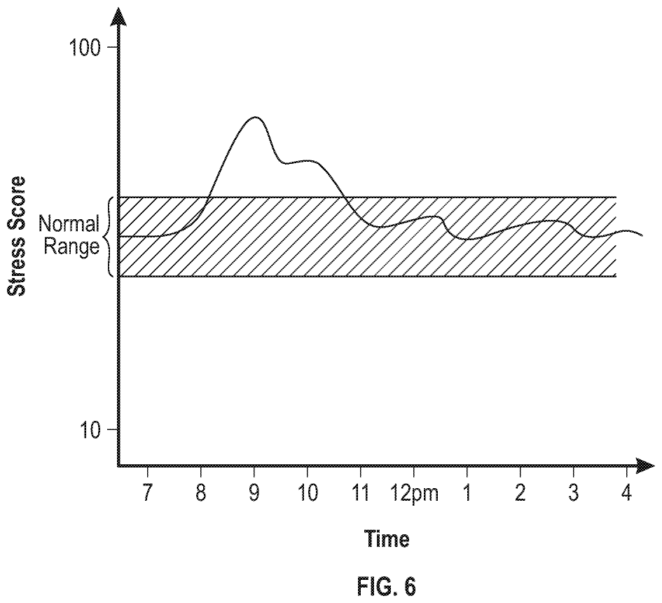

FIG. 6 illustrates a graphical representation of a stress reading in accordance with an embodiment of the present subject matter. The graph shows a stress reading for a user throughout the day based on the physiological conditions monitored, as described herein. The graph may display a normal range of stress readings. Stress readings above the normal range may be categorized as high or elevated values and stress readings below the normal range may be categorized as low or decreased values. As shown in this example, the user had high stress readings from approximately 8 am to 10 am, and normal stress readings for the rest of the day. Thus, the user may be able to isolate what triggered the higher stress readings between 8 am to 10 am. For example, a patient of the user (e.g., a healthcare provider) may be the cause of the increased stress in the user during the 8 am to 10 am time period. The user may then be able to take actions to reduce his/her stress during interactions with that particular patient, who was the cause of the additional stress.

In an embodiment, the reference values may be customized or personalized for the user. In this embodiment, reference values for the physiological indicator(s) may be obtained from the user during a non-stressful time (say, a weekend). Thus, the received sensor information may then be compared to the personal reference values of the user to offer a more customized evaluation of the stress level of the user. This may be particularly helpful for patients with underlying conditions, which might elevate or suppress certain readings.

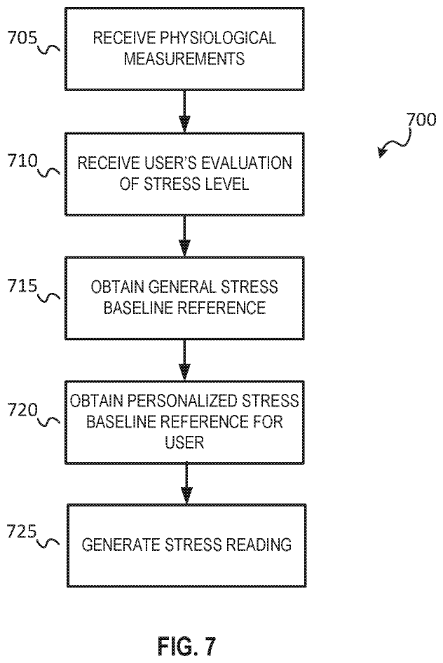

FIG. 7 illustrates a flow diagram of a method 700 to generate a stress reading in accordance with an embodiment of the present subject matter. Method 700 may be executed by an EHR server as described herein. At 705, physiological measurements may be received. The physiological measurements may be procured using a plurality of sensors as described herein. For example, these physiological measurements may include pupil diameter, voice, cortisol levels, skin resistance, etc. The physiological measurements may be filtered to remove noise. Even if sensors are properly placed, the raw physiological data can include small fluctuations caused by oscillations or inconsistencies of the physiological status of human bodies. Noise may be filtered using a variety of signal processing techniques, such as Kalman filter, Butterworth filter, Median filter, Weiner filter, Wavelet decomposition, etc. The selection of the signal processing technique (e.g., filter) may be based on the nature of the signal and features to be extracted and types of noise. Thus, a different signal processing technique may be used for different physiological measurements to remove noise from the respective data.

Moreover, the physiological measurements may be timestamped and include geolocation data. The timestamp and/or geolocation may be added by respective sensors. This information may then be used later to identify time and/or location of moments of stress.

At 710, a subjective valuation by the user of his/her stress level may be received. A user may input his/her perception of his/her current stress level into a user device, and this valuation may be forwarded to the EHR server. For example, a user may be prompted to select his/her perceived current stress level on a scale of 1-10, 1 being least stressed and 10 being most stressed. In another embodiment, the subjective valuation may be calculated from other indirect indicators of stress. For example, a user may be prompted to input his/her perceived current mood and/or anxiety levels. The EHR server may then calculate the subjective valuation of the user's stress level from the mood and/or anxiety levels.

At 715, a general baseline reference for the stress level for the user may be obtained. As described herein, the general baseline reference may be based on the age group of the user or other general reference information.

At 720, a personalized baseline reference for the user may be obtained. The personalized baseline reference may be determined using historical data of the user. For example, it may be determined from procuring physiological measurements of the user in designated non-stressful situations. For example, the physiological measurements may be obtained during a weekend, a vacation, an evening, deep sleep, etc. This personalized baseline reference may represent physiological measurements taken during known non-stressful conditions. Thus, the personalized baseline reference may provide a customized baseline reference for comparison for the user, which in some cases may be more useful than the general baseline reference.

At 725, a stress reading may be generated. The stress reading may be generated using the information collected in one or more of steps 705-720. For example, the stress reading may be generated using physiological measurements received from the sensor(s), the subjective valuation of the user's stress level, the general baseline reference for the user, and/or the personalized baseline reference for the user. Appropriate weights may be applied to different physiological measurements, based on the user and/or circumstances.

In one example, the following physiological measurements may be received: GSR, voice, blood pressure (oxygen), pulse, heart rate variability, sleep and R-R variability. For this user, these physiological parameters may be weighted equally. Based on these physiological measurements, a stress reading may be generated. This stress reading may take into account a previously generated stress baseline reference for that user. This stress reading may also take into account a subjective valuation of the user of his/her stress during that time. Moreover, the stress reading may take into account a general stress baseline reference for that user.

In an embodiment, when any measured parameter reaches a set point for the user, the user may receive an immediate warning. This warning may allow the user to utilize destressing techniques, such as those learned through biofeedback. Based on the measured parameter(s), the system may determine whether induced stress emotional or cognitive stress and provide the user with specific feedback based on the type of stress.

Physiological parameters may be measured in real time, and biofeedback may be provided in real time, too, allowing the user to utilize techniques to reduce his/her stress level. The techniques may include meditation, going for a walk, deep breathing, controlled breathing, listing to calm music, etc. The biofeedback may indicate whether those destressing techniques are effective. For example, the user device (e.g., phone app, watch, computer) may indicate current stress level of the user by color, e.g., red, yellow, green, blue may indicate different levels of stress with red being the highest and blue being the lowest amount of detected stress. Also, the user may receive an alarm if the user's stress reading exceeds a threshold to alert the user to initiate a destressing technique. The biofeedback may provide instant indication of whether the destressing technique is working.

In an embodiment, some periods may be excluded from stress reading measurements. For example, periods where the user is exercising and sometime thereafter (e.g., 45 minutes) may be excluded from stress reading measurement. For example, if exercise is detected based on elevated heart, accelerometer reading, etc., that period of time (and sometime thereafter) may be excluded from stress measurements.

In some situations, one or more physiological measurements may not be relevant for indicating stress levels, and in these situations, those measurements may be excluded from generating the stress reading. For example, when the user is exercising, some of the user's physiological measurements may become elevated due to exercising, not stress, such as heart rate, respiratory rate, sweat, etc. In those instances, the EHR server may exclude the physiological measurements affected by the user exercising.

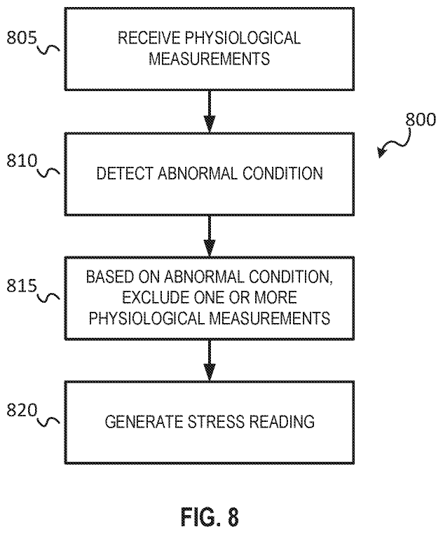

FIG. 8 illustrates a flow diagram of a method 800 to calculate a stress reading during an abnormal condition in accordance with an embodiment of the present subject matter. Method 800 may be executed by an EHR server as described herein. At 805, physiological measurements may be received. The physiological measurements may be procured using a plurality of sensors as described herein.

At 810, based on the received physiological measurements, an abnormal condition (e.g., exercise, sleep) may be detected. For example, based on elevated heart rate, sweat, respiratory rate, etc., the abnormal condition may be detected. In another embodiment, a user may alert the system of the abnormal condition. For example, the user may send a message or notification, alerting the EHR server of an upcoming abnormal condition, such as before the user begins exercising. In another embodiment, the abnormal condition, such as exercise, may be detected by other sensors such as an accelerometer.

At 815, based on the type of detected abnormal condition, one or more physiological measurements may be excluded. If the detected abnormal condition is exercise, physiological measurements for heart rate, respiratory rate, and sweat may be excluded. At 820, a stress reading may be generated using other physiological measurements, as described herein, but without the excluded physiological measurements. In an embodiment, the detected times of exercise and other activities may be labeled on a graphical display to the user.

The data described herein may be encrypted. Moreover, the data and user may be anonymized. That is, separate from encryption, the data and user may be anonymized so that, user identity may be hidden. Anonymization may be performed by assigning random identifiers to the users and the data may be tracked using the random identifiers. In an embodiment, the random identifier may change with each session the user logs onto the system.

Furthermore, the EHR server may also provide resource materials for users to assist them with dealing with stress. For example, the EHR server may provide reference information for professionals who can help the user with managing their stress. The listing of the reference information may be filtered by the location of the user.

### Stress Score Example

In this example, at least seven physiological parameters may be utilized: heart rate variability, voice analysis, heart rate, blood pressure, sleep, respiratory rate, and GSR. More or less physiological parameters may be used, as described herein. Stress readings may be determined as an average over a 24-hour period to represent stress over the course of a particular day. The stress readings may be provided on a coordinate grid with the y-axis representing stress intensity on a scale (e.g., 1-100) and the x-axis may represent time. This graphical representation may allow users to correlate stressful moments to particular temporal events. Moreover, for a specified time, such as the current time, a stress reading/score may be provided in real time (e.g., on a scale of 1-100), as an average of the physiological parameters at that time. The parameters may be weighted differently.

The results may be provided in two grids. A first grid may represent ranges of personalized scores after establishing a personal stress baseline level. This grid may compare the user's stress reading to their own personal baseline, which may be established in relaxed states to stressed states over the course of the day.

A second grid may compare the user's stress reading to established physiological parameters of healthy humans in the same group as the user (e.g., age group). This may allow the user to understand how they compare to cognitively and physically healthy individuals.

Other scores may be determined in addition to the stress reading. A physical fitness score may be determined. For example, the physical fitness score may be determined based on heart rate variability, heart rate, and blood pressure. Each of these parameters may be weighted equally in determining the physical fitness score (e.g., 33% each). These values may be comparative values in relation to healthy subjects in the same group as the user. Physically fit people tend to cope with stress better than unfit individuals. In an example, feedback may be provided to users who test low on the physical fitness score to encourage them to exercise more and to provide other destressing techniques.

A cognitive fitness score may be determined. For example, the cognitive fitness score may be determined based on voice analysis, respiration, and sleep. Each of these parameters may be weighted equally in determining the cognitive fitness score (e.g., 33% each). These values may be comparative values in relation to healthy subjects in the same group as the user. Individuals with anxiety and mood disorders tend to cope with stress worse than others, even when dealing with usual daily life stressors. In an example, feedback may be provided to users who test low on the cognitive fitness score to encourage them to use destressing techniques, such as meditating, to use biofeedback techniques. In addition users will be provided links to connect with mental health providers through the EHR.

Different displaying techniques may be used. For example, individual parameters may be displayed separately in a color-coded format on the same coordinated grid. This may allow users to compare different parameters over the course of the day to each other. This may also allow detection of other conditions. For example, if blood pressure is detected to be high while the pulse rate is detected to be low, this may be indicative of hypertension. Conversely, if pulse rate is detected to be high while blood pressure is detected to be low, this may be indicative of anxiety.

Different alarms or indications may be used. Users may be given options to receive an alert when their stress readings cross a certain threshold, which may be enable them to institute destressing techniques individually. Users may also set alerts for individual parameters.

The measured parameters may be weighted differently when determining stress readings. In one example, voice analysis and heart rate variability may be weighted the most. Heart rate, blood pressure, sleep and respiration may be weighted less than voice analysis and heart rate variability while more than GSR. For example, voice analysis and heart rate variability may each represent 20% of the stress reading score; heart rate, blood pressure, sleep, and respiration may each represent 13% of the stress reading score; and GSR may represent 8% of the stress reading score.

Heart rate variability may be indicative of stress events. In individuals, low heart rate variability is typically less than 23.9 milliseconds; intermediate heart rate variability is between 23.9 and 35.4 milliseconds; and high heart rate variability is above 35.4 milliseconds. These values correspond inversely to stress. Heart rate variability typically decreases with age. Hence, standardized values for heart rate variability may be provided based on the age of the user. Moreover, the baseline for individual heart rate variability may be measured during non-stressful times such as during sleep or a relaxed day. After a 24-hour baseline reference for heart rate variability is established, comparisons may be made to it for stress monitoring. For example, a one millisecond decrease in heart rate variability in a given time (e.g., 24 hours) may represent a 10% increase in the heart rate variability score.

Regarding voice analysis, a normal fundamental frequency (f0) tends to increase with stress. For men, a standard score for normal fundamental frequency is between 85-180 Hz with an average of 120 Hz. For women, it is between 165-255 HZ with an average of 210 Hz. The baseline for a user's normal fundamental frequency may be measured at during non-stressful times such a relaxed day. After establishing a baseline, 1% increments in a given time (e.g., 24 hours) of f0 may represent a 3% increase of the voice score.

Regarding heart rate, a normal resting heart rate for an adult is 72 beats per minute. The baseline for a user's heart rate may be measured during non-stressful times such as during sleep or a relaxed day. After establishing a baseline, 1 beat per minute increase in a given time (e.g., 24 hours) may correspond to a 7% increase on the heart rate score.

Regarding blood pressure, a normal blood pressure is typically 120/80 mm hg. The upper value represents systolic blood pressure (the maximum pressure your heart exerts while beating). Stress can increase systolic blood pressure. The baseline for blood pressure for a user may be measured during non-stressful times such as during sleep or a relaxed day. After establishing a baseline, an increase of 1 mm hg over a given time (e.g., 24 hours) may represent a 6% increase in the blood pressure score.

Regarding respiration, respiratory rate and variability may be measured. Normal respiratory rate is 12-20 breaths per minute. Research shows that different emotions are associated with different forms of breathing, so changes in breathing patterns may be indicative of emotional states. For example, joy can elicit regular, deep, and slow breathing. Anxiety or anger can change breathing patterns to be irregular, short, fast and shallow. Breathing rate and inter-breath variability may be weighted equally (e.g., 50% each). The baseline for respiration may be measured during non-stressful times such as during sleep or a relaxed day. After establishing a baseline, increases in breathing rate and an increase of one breath per minute (breathing rate) may represent 16.6% of the breathing rate score, and a change of inter-breath variability of 0.25 seconds may represent an 8.3% increase of inter-breath variability score.

Regarding sleep, an average restful sleep duration falls between 7-9 hours. Even slight sleep deprivation or poor sleep can affect memory, judgment, and mood. Stress tends to lead to sleep deprivation. The baseline for sleep duration may be measured during a relaxed, un-stressful period (e.g., vacation or weekend). After establishing a baseline, a decrease in 15 minutes of sleep duration may represent a 12.5% increase in the sleep score.

Regarding GSR, normal GSR is between 10-50 micro-Siemens. Low GSR is indicative of stress and high GSR is indicative of a relaxed state. The baseline for GSR may be measured during non-stressful times such as during sleep or a relaxed day. After establishing a baseline, a decrease of 1 micro-Siemens over a given time (e.g., 24 hours) may represent a 10% increase in the GSR score.

The monitoring techniques described herein may have different applications, too. For example, the monitoring techniques described herein may be used for different management techniques.

### Bipolar Disorder Management Example

Bipolar affective disorder is one of the leading causes of disability in the United. States and globally. It is prevalent in at least 4% of the population. Early detection of a mania episode may improve management of this disorder and decrease the incidence of morbidity and mortality.

In this example, at least four parameters may be utilized for detecting mania: sleep duration, speech, activity level, and risky driving. More or less parameters may be used. Each parameter may be weighted equally in determining a total mania score (e.g., 25% each).

These parameters will be monitored (for patients and providers), via an app on their cellphone, wearable device, computer, etc., as described herein.

Baseline values for sleep duration, speech (rate and amplitude), activity level, number of steps a day, time spent on phone and electronic devices and risk-taking behavior such as driving fast may be calculated during a one-week period of euthymia (normal mood). This one-week period may be validated by the individual's health care providers such as their therapist. Medical providers may be provided access to these scores (for their patients), using the EHR system. Above threshold readings may prompt a warning or alert to the patient, their provider, and/or other trusted people.

During period of euthymia, users may be monitored continuously. Hence, users may be instructed to keep their phone, wearable devices, etc., on them at substantially all times. This may involve using a plurality of monitoring devices to compensate for night, charging times, etc. If devices are not detected as being worn or carried, this may indicate a manic state and may prompt an alert.

In general, values above 40% may prompt an alert to the patient's health care provider and/or other trusted people. These levels may be set lower by the provider in cases where patients have more severe conditions. Providers may be provided access to readings and may incorporate these measurements during visits. Measured parameters will be provided on a coordinate grid with intensity of symptoms on the Y axis and the X axis representing time. Moreover, the parameters may be compared to euthymic personal baselines.

Regarding sleep, an average restful sleep duration falls between 7-9 hours. The baseline for sleep duration may be measured during a euthymic period (e.g., a week certified by medical care provider). After establishing a baseline, a decrease in 15 minutes of sleep duration may represent a 16.6% increase in the sleep score.

Regarding speech, average words per minute (wpm) for English speakers is 120-150 wpm, and average speech amplitude is 60 dB. Average wpm and speech amplitude may be weighted differently. For example, wpm may account for 75% of the total speech score while speech amplitude may account for 25% of the total speech score. The baseline for wpm and speech amplitude may be measuring during a euthymic period. After establishing a baseline, an average increase in 10 wpm may represent a 30% increase of the wpm score and an average increase in 1 dB may represent a 10% increase in the speech amplitude score.

Activity level may be based on physical and mental activity. Physical activity may be measured by a pedometer (steps per day) via the wearable device, phone, etc. This physical activity measurement may represent 50% of the total activity level score. Mental activity may be measured by monitoring time spent on devices such as phone (e.g., online activity in minutes). This mental activity measurement may represent 50% of the total activity score. The baseline for activity levels may be measuring during a euthymic period.

Erratic or reckless driving may also be indicative of mania. Driving may be monitored based on speed, acceleration, etc. For example, speed may represent 75% of the driving score and the amount of time spent driving above the speed limit may represent 25% of the driving score. The baseline for driving behavior may be measuring during a euthymic period. In one example, after establishing the baseline, each 5 mph over the speed limit on a highway may represent a 16.6% increase of the speed score and each 1 mph over the speed limit on city roads may represent a 6.6% increase of the speed score. The user's devices may be used to monitor the speed and location (e.g., highway, city road) of the user. Regarding amount of time, after establishing the baseline, each 20 seconds (or other specified time period) driving at least 15 mph over the speed limit on the highway and 5 mph over the speed limit on city roads may represent a 20% of the amount of time score.

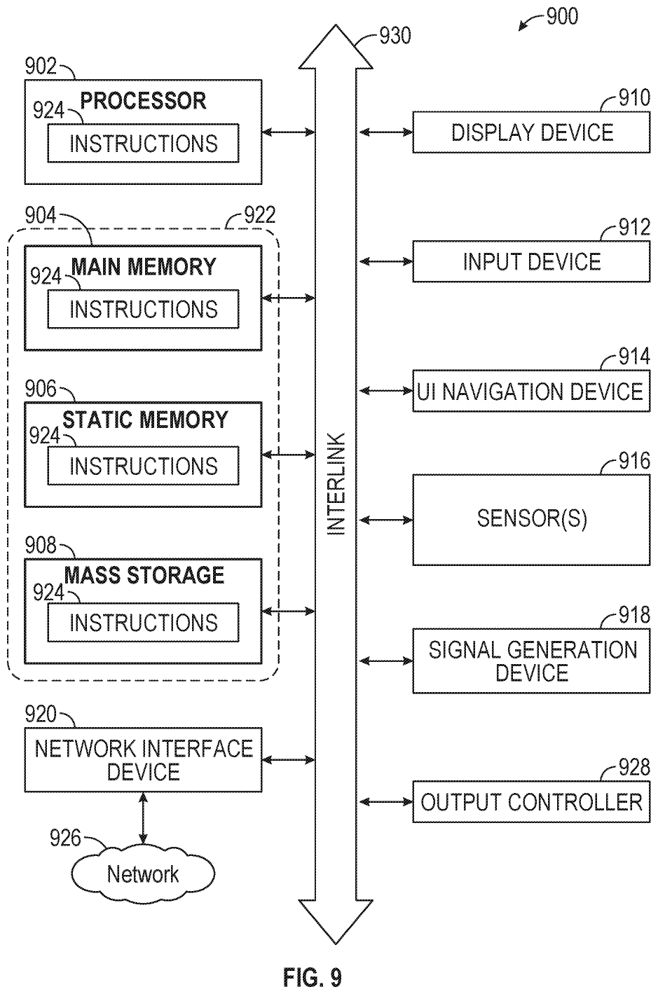

The techniques shown and described in this document can be performed using a portion or an entirety of stress monitoring system 100 as shown in FIG. 1 or otherwise using a machine 900 as discussed below in relation to FIG. 9.

FIG. 9 illustrates a block diagram of an example comprising a machine 900 upon which any one or more of the techniques (e.g., methodologies) discussed herein may be performed. In various examples, the machine 900 may operate as a standalone device or may be connected (e.g., networked) to other machines. In a networked deployment, the machine 900 may operate in the capacity of a server machine, a client machine, or both in server-client network environments. In an example, the machine 900 may act as a peer machine in peer-to-peer (P2P) (or other distributed) network environment. The machine 900 may be a personal computer (PC), a tablet device, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuitry is a collection of circuits implemented in tangible entities that include hardware (e.g., simple circuits, gates, logic, etc.). Circuitry membership may be flexible over time and underlying hardware variability. Circuitries include members that may, alone or in combination, perform specified operations when operating. In an example, hardware of the circuitry may be immutably designed to carry out a specific operation (e.g., hardwired). In an example, the hardware comprising the circuitry may include variably connected physical components (e.g., execution units, transistors, simple circuits, etc.) including a computer readable medium physically modified (e.g., magnetically, electrically, such as via a change in physical state or transformation of another physical characteristic, etc.) to encode instructions of the specific operation. In connecting the physical components, the underlying electrical properties of a hardware constituent may be changed, for example, from an insulating characteristic to a conductive characteristic or vice versa. The instructions enable embedded hardware (e.g., the execution units or a loading mechanism) to create members of the circuitry in hardware via the variable connections to carry out portions of the specific operation when in operation. Accordingly, the computer readable medium is communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time.

Machine (e.g., computer system) 900 may include a hardware processor 902 (e.g., a central processing unit (CPU), a graphics processing unit (GPU), a hardware processor core, or any combination thereof), a main memory 904 and a static memory 906, some or all of which may communicate with each other via an interlink (e.g., bus) 908. The machine 900 may further include a display unit 910, an alphanumeric input device 912 (e.g., a keyboard), and a user interface (UI) navigation device 914 (e.g., a mouse). In an example, the display unit 910, input device 912 and UI navigation device 914 may be a touch screen display. The machine 900 may additionally include a storage device (e.g., drive unit) 916, a signal generation device 918 (e.g., a speaker), a network interface device 920, and one or more sensors 921, such as a global positioning system (GPS) sensor, compass, accelerometer, or other sensor. The machine 900 may include an output controller 928, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 916 may include a machine readable medium 922 on which is stored one or more sets of data structures or instructions 924 (e.g., software) embodying or utilized by any one or more of the techniques or functions described herein. The instructions 924 may also reside, completely or at least partially, within the main memory 904, within static memory 906, or within the hardware processor 902 during execution thereof by the machine 900. In an example, one or any combination of the hardware processor 902, the main memory 904, the static memory 906, or the storage device 916 may constitute machine readable media.

While the machine readable medium 922 is illustrated as a single medium, the term “machine readable medium” may include a single medium or multiple media (e.g., a centralized or distributed database, and/or associated caches and servers) configured to store the one or more instructions 924.

The term “machine readable medium” may include any medium that is capable of storing, encoding, or carrying instructions for execution by the machine 900 and that cause the machine 900 to perform any one or more of the techniques of the present disclosure, or that is capable of storing, encoding or carrying data structures used by or associated with such instructions. Non-limiting machine readable medium examples may include solid-state memories, and optical and magnetic media. Accordingly, machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic or other phase-change or state-change memory circuits; magnetic disks, such as internal hard disks and removable disks; magneto-optical disks; and CD-ROM and DVD-ROM disks.

The instructions 924 may further be transmitted or received over a communications network 926 using a transmission medium via the network interface device 920 utilizing any one of a number of transfer protocols (e.g., frame relay, Internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 920 may include one or more physical jacks (e.g., Ethernet, coaxial, or phone jacks) or one or more antennas to connect to the communications network 926. In an example, the network interface device 920 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing; encoding or carrying instructions for execution by the machine 900, and includes digital or analog communications signals or other intangible medium to facilitate communication of such software.

### Various Notes

Each of the non-limiting aspects above can stand on its own or can be combined in various permutations or combinations with one or more of the other aspects or other subject matter described in this document.

The above detailed description includes references to the accompanying drawings, which form a part of the detailed description. The drawings show, by way of illustration, specific implementations in which the present subject matter can be practiced. These implementations are also referred to generally as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other implementations can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed implementation. Thus, the following claims are hereby incorporated into the Detailed Description as examples or implementations, with each claim standing on its own as a separate implementation, and it is contemplated that such implementations can be combined with each other in various combinations or permutations. The scope of the invention should be determined with reference to the appended claims, along with the full scope of equivalents to which such claims are entitled.

The following numbered examples are embodiments:

Example 1. A method comprising: receiving a plurality of physiological measurements associated with a user for a time period; retrieving a personalized stress baseline for the user, wherein the personalized stress baseline is determined using physiological measurements obtained at a designated non-stressful time; based on the received plurality of physiological measurements and personalized stress baseline, generating a stress reading; and transmitting information associated with the generated stress reading to the user.

Example 2. The method of example 1, further comprising: retrieving a general stress baseline for the user, wherein the general stress baseline is based on the age of the user; and wherein the generated stress reading is based on the general stress baseline

Example 3. The method of any of examples 1-2, further comprising: receiving a user evaluation of a stress level of the user; wherein the generated stress reading is based on the user evaluation of the stress level.

Example 4. The method of any of examples 1-3, further comprising: detecting an abnormal activity during the time period; and based on detecting the abnormal activity, excluding at least one of the plurality of physiological measurements from the stress reading generation.

Example 5. The method of any of examples 1-4, further comprising: detecting an abnormal activity during a portion of the time period; and based on detecting the abnormal activity, cease generating the stress reading for the portion of the time period.

Example 6. The method of any of examples 1-5, further comprising: weighting the plurality of physiological measurements.

Example 7. The method of any of examples 1-6, wherein the plurality of physiological measurements provide information about heart rate, voice, and respiration.

Example 8. A system comprising: one or more processors of a machine; and a memory storing instructions that, when executed by the one or more processors, cause the machine to perform operations implementing any one of example methods 1 to 7.

Example 11. A machine-readable storage device embodying instructions that, when executed by a machine, cause the machine to perform operations implementing any one of example methods 1 to 7.

What is claimed is:

1. A method comprising:
receiving a plurality of physiological measurements associated with a user for a time period;
retrieving a personalized stress baseline for the user, wherein the personalized stress baseline is determined using physiological measurements obtained at a designated non-stressful time;
based on the received plurality of physiological measurements and personalized stress baseline, generating a stress reading; and
transmitting information associated with the generated stress reading to the user.

2. The method of claim 1, further comprising:
retrieving a general stress baseline for the user, wherein the general stress baseline is based on the age of the user; and
wherein the generated stress reading is based on the general stress baseline.

3. The method of claim 1, further comprising:
receiving a user evaluation of a stress level of the user;
wherein the generated stress reading is based on the user evaluation of the stress level.

4. The method of claim 1, further comprising:
detecting an abnormal activity during the time period; and
based on detecting the abnormal activity, excluding at least one of the plurality of physiological measurements from the stress reading generation.

5. The method of claim 1, further comprising:
detecting an abnormal activity during a portion of the time period; and
based on detecting the abnormal activity, cease generating the stress reading for the portion of the time period.

6. The method of claim 1, further comprising:
weighting the plurality of physiological measurements.

7. The method of claim 1, wherein the plurality of physiological measurements provide information about heart rate, voice, and respiration.

8. A stress monitoring system comprising:
one or more processors of a machine; and
a memory storing instructions that, when executed by the one or more processors, cause the machine to perform operations comprising:
receiving a plurality of physiological measurements associated with a user for a time period;
retrieving a personalized stress baseline for the user, wherein the personalized stress baseline is determined using physiological measurements obtained at a designated non-stressful time;
based on the received plurality of physiological measurements and personalized stress baseline, generating a stress reading; and
transmitting information associated with the generated stress reading to the user.

9. The stress monitoring system of claim 8, the operations further comprising:
retrieving a general stress baseline for the user, wherein the general stress baseline is based on the age of the user; and
wherein the generated stress reading is based on the general stress baseline.

10. The stress monitoring system of claim 8, the operations further comprising:
receiving a user evaluation of a stress level of the user;
wherein the generated stress reading is based on the user evaluation of the stress level.

11. The stress monitoring system of claim 8, the operations further comprising:
detecting an abnormal activity during the time period; and
based on detecting the abnormal activity, excluding at least one of the plurality of physiological measurements from the stress reading generation.

12. The stress monitoring system of claim 8, the operations further comprising:
detecting an abnormal activity during a portion of the time period; and
based on detecting the abnormal activity, cease generating the stress reading for the portion of the time period.

13. The stress monitoring system of claim 8, the operations further comprising:
weighting the plurality of physiological measurements.

14. The stress monitoring system of claim 8, wherein the plurality of physiological measurements provide information about heart rate, voice, and respiration.

15. A machine-storage medium embodying instructions that, when executed by a machine, cause the machine to perform operations comprising:
receiving a plurality of physiological measurements associated with a user for a time period;
retrieving a personalized stress baseline for the user, wherein the personalized stress baseline is determined using physiological measurements obtained at a designated non-stressful time;
based on the received plurality of physiological measurements and personalized stress baseline, generating a stress reading; and
transmitting information associated with the generated stress reading to the user.

16. The machine-storage medium embodying instructions of claim 15, further comprising:
retrieving a general stress baseline for the user, wherein the general stress baseline is based on the age of the user; and
wherein the generated stress reading is based on the general stress baseline.

17. The machine-storage medium embodying instructions of claim 15, further comprising:
receiving a user evaluation of a stress level of the user;
wherein the generated stress reading is based on the user evaluation of the stress level.

18. The machine-storage medium embodying instructions of claim 15, further comprising:
detecting an abnormal activity during the time period; and
based on detecting the abnormal activity, excluding at least one of the plurality of physiological measurements from the stress reading generation.

19. The machine-storage medium embodying instructions of claim 15, further comprising:
detecting an abnormal activity during a portion of the time period; and
based on detecting the abnormal activity, cease generating the stress reading for the portion of the time period.

20. The machine-storage medium embodying instructions of claim 15, further comprising:
weighting the plurality of physiological measurements.
